(19) United States
(12) Patent Application Publication
(10) Pub. No.: US 2003/0165483 A1
(43) Pub. Date: Sep. 4, 2003

(54) Method for the modification of biological cells

(76) Inventors: Ulrich Zimmermann, Waldbrunn (DE); Kurt Lucas, Hamburg (DE); Ulrich Zimmermann, Waldbrunn (DE); Kurt Lucas, Hamburg (DE); Barbara Köppen, Hamburg (DE)

Correspondence Address: Matthew P McWilliams, Buchanan Ingersoll, Eleven Penn Center 14th Floor, 1835 Market Street, Philadelphia, PA, 19103-2895, US

(21) Appl. No.: 10/332,636

(22) PCT Filed: Jul. 6, 2001
(86) PCT No.: PCT/EP01/07789

(30) Foreign Application Priority Data
Jul. 10, 2000 (DE) 10033470.9
Oct. 30, 2000 (DE) 10053783.9
Feb. 28, 2001 (DE) 10109618.6

(51) Int. Cl. A61K 48/00

(52) U.S. Cl. 424/93.21; 424/93.7

(57) ABSTRACT

A method of modifying antigen-presenting cells is described, in which antigens of diseased cells or their cell components are transferred to the antigen-presenting cells, the antigen-presenting cells being brought into contact with the diseased cells or their cell components on a solid-phase substrate or in a suspension through introduction into a shared suspension and exertion of external forces, membrane components having antigens without cell nuclei of the diseased cells or their cell components being transferred to the antigen-presenting cells and subsequently a separation of the modified cells from the solid-phase substrate or from the suspension being performed.

The invention relates to methods of handling and/or treating biological and/or synthetic cells, particularly for the interaction of cells or cell components which represent or carry antigens (e.g., tumor cells), and cells which present antigens (such as mast cells or dendritic cells), methods of producing compounds from antigen-presenting cells and diseased cells, for example, tumor cells, or their cell components (e.g., membrane parts), methods of modifying antigen-presenting cells, for example, dendritic cells, with diseased cells, for example, tumor cells, or their cell components (e.g., membrane parts), methods of passive immunization or inoculation of organisms against diseases (e.g., viral or bacterial infections, tumor diseases, or the like), methods of treating infectious or tumor diseases and/or methods of producing compounds for passive immunization or inoculation of organisms against infectious or tumor diseases, devices for implementing the methods, applications of devices for dielectrophoretic manipulation of cells for modifying antigen-presenting cells, particularly dendritic cells or mast cells, and applications of the methods cited.

For tumor treatment, there have been attempts to subject an organism to excitation of the immune system, in that foreign dendritic cells or those from the organism are modified using antigens and supplied to the organism. The dendritic cells act as immunoactive cells, which have immunostimulating properties as a function of the respective antigens. The formation and properties of dendritic cells are, for example, described by K. Shortman et al. in “Stem Cells”, Vol. 15, 1997, p. 409 et seq. The modification of the dendritic cells using antigens means that the antigens are incorporated into the surface of the dendritic cells. For example, specific model peptides (see P. Paglia et al. in “Minerva Biotecnologica”, Vol. 11, 1999, p. 261 et seq.) or antigens formed by the respective tumor cells are used as antigens. To charge the dendritic cells with antigens of tumor cells, the following methods are known.

In the method described, for example, by T. H. Scott-Taylor et al. in “Biochimica et Biophysica Acta”, Vol. 1500, 2000, p. 265 et seq., using electrofusion, tumor cells are fused together with dendritic cells, which may particularly absorb the tumor antigens with the tumor cells and thus trigger an excitation of the immune system. This method has the disadvantage that electrofusion is a complex process, which requires application-specific optimization of the fusion conditions. The electrofusion of dendritic cells with tumor cells also has the disadvantage that the yields described in the literature are very low and, due to the random combination of the genes of the two fusion partners, fusion products having different properties result, which may lead to different immune reactions after their return into the patients. In order to not stress the organism to be treated with even more tumor cells, the tumor cells must be killed, using radioactive irradiation, for example, before the electrofusion. However, a risk remains in the typical irradiation methods that tumor cells will survive and lead to metastases. Therefore, immunization methods which are based on the electrofusion of entire cells have only restricted reliability, since it must be ensured that the tumor cells are all killed, and that all pathogens which are found in the tumor cells, such as viruses, are deactivated.

The use of tumor cell lysates to modify the dendritic cells is known from the publication of K. Shimizu et al. in “Proc. Natl. Acad. Sci. USA”, Vol. 96, 1999, p. 2268. The lysates are tumor cells which have been destroyed by a freezing and thawing process. The interaction of the dendritic cells with the lysates is induced by a long-term incubation over approximately 20 hours. In addition to the large expenditure of time, the use of the lysates has the disadvantage of restricted reliability. As in electrofusion, radioactive irradiation is provided in order to reduce the danger of additional metastasis.

The production of an association of dendritic cells and tumor cells is also described by C. M. Celluzzi et al. in “The Journal of Immunology”, Vol. 160, 1998, p. 3081 et seq. The association is performed either through electrically stimulated cell fusion or through physical interaction during long-term incubation, which leads to a so-called mock fusion. The problems cited above also arise for this method. The cell association used to trigger the immune reaction carries a significant risk of metastasis in itself with the nucleus of the tumor cell. In addition, the production of mock fusions requires a time-consuming co-incubation having a duration of more than 10 hours.

A physical method of introducing DNA molecules into cells using accelerated solid particles (microprojectiles) is known from the publication of N.-S. Yang et al. (“Proc. Natl. Acad. Sci. USA” Vol. 87, 1990, p. 9568 et seq. ”). The microprojectiles are coated gold or tungsten particles with a size of 1 μm. The DNA is bonded to the particle surface. The bombardment, at speeds in the range of 500 m/sec, is performed using pressure sources or electric fields. This method of DNA transfection is also referred to as biolistic particle bombardment.

The object of the present invention is to provide novel methods of treating biological cells in which cells are caused to interact with one another. In particular, an improved method of modifying dendritic cells is to be provided, using which the disadvantages of typical techniques are overcome and which is distinguished by a rapid method cycle and the exclusion of a risk of metastasis. The method according to the present invention is also to be improved in regard to the reproduceability of the setting of interaction conditions. The object of the present invention is also to provide devices for performing the method and applications of the method. Furthermore, a novel, expanded method of modifying cells in suspensions is to be provided.

A first basic idea of the present invention is to cause biological and/or synthetic cells, cell components, or macromolecules to interact with one another, in that the respective particles are brought into contact with one another, the cells, cell components, or macromolecules caused to interact being in a suspended state in a suspension. A modification of dendritic cells or other antigen-presenting cells with diseased cells is performed in the suspended state. Antigen-presenting cells and diseased cells or cell components of diseased cells (e.g., tumor cells or tumor cell components, bacteria, viruses/virus envelopes, stem cells, bone marrow cells, or epithelial cells) are subjected to external forces in a suspension in such a way that the various cell types or cell components mutually contact one another. The inventor has determined that contact under the effect of external forces is sufficient to transfer exclusively membrane components having the antigens of the diseased cells or cell components to the antigen-presenting cells. Cell nuclei and other components of the diseased cells remain in the suspension. The modified antigen-presenting cells are separated from the still free diseased cells or cell components in the suspension and made available for the respective application, particularly the excitation of the immune system of an organism. According to a preferred aspect of the present invention, the method is used for generating immunologically active cells, particularly immunologically active dendritic cells. A subject of the present invention is particularly the modification of suspended cells presenting antigens, such as T-cells, B-cells, or mast cells.

In the interaction of the cells or cell components, in general, every type of mechanical or material or other interaction, particularly interactions in which a substance transfer or a transfer of cell components onto a dendritic cell, for example, occurs, is considered. The cells are coupled through the formation of chemical bonds or physical associations, so that the cell membranes come into contact. When the cell membranes come into contact, a mutual interaction occurs. For example, membrane pieces of one cell type are transferred onto the other cell type.

A further basic idea of the invention is to cause biological and/or synthetic cells, cell components, or macromolecules to interact with one another, in that the respective particles are brought into contact with one another, at least one group of the cells, cell components, or macromolecules caused to interact being in an adherent state on a solid-phase substrate. The solid-phase substrate is formed by a planar substrate, for example, a substrate having at least one glass, plastic, or membrane surface, or a particulate substrate. The solid-phase substrate having the adherent cells is introduced into a liquid in which the respective other group of cells, cell components, or macromolecules is suspended. The suspended particles are subjected to external forces in the suspension in such a way that the various cell types or cell components come into contact with one another. The inventors have determined that contact under the effect of external forces is sufficient to transfer exclusively membrane components having the antigens of the diseased cells or cell components onto the antigen-presenting cells, for example, dendritic cells. Cell nuclei and other components of the diseased cells or cell components remain in the suspension.

According to a preferred embodiment of the present invention, the antigen-presenting cells are bonded to the solid-phase substrate, which is positioned in the suspension liquid. The diseased cells or cell components, from which antigens are to be transferred onto the adsorbed cells, are added to the suspension. After the mutual interaction, the modified adsorbed cells are separated from the still free diseased cells or cell components in the suspension and made available for the respective application, particularly the excitation of the immune system of an organism. The separation is preferably performed by removing the diseased cells or cell components from the suspension or transferring the solid-phase substrate into another environment. Subsequently, the modified cells are detached from the solid-phase substrate. This is performed, for example, through an enzymatic process. According to a preferred aspect of the present invention, the method is used for generating immunologically active dendritic cells.

According to a modified embodiment of the present invention, the antigen-presenting cells are brought into contact with diseased cells in the adsorbed state, so that antigens of the diseased cells are transferred onto the antigen-presenting cells. The transfer of antigens again occurs in that membrane components of the diseased cells adhere to the antigen-presenting cells or are incorporated or absorbed into their membranes.

A decisive advantage of the present invention is that the modification of the antigen-presenting cells may be performed within short times like in conventional electrofusion, however, in contrast to the conventional fusion or mock fusion techniques, only cell components having the desired antigens, but not entire tumor cells, are advantageously coupled to the dendritic cells. In this way, the risk of metastasis upon application of the dendritic cells is eliminated for the first time.

The binding of the group of the cells transferring the antigens or the group of the antigen-presenting cells to a solid-phase substrate has the additional advantage that the parameters of the mutual interaction, particularly forces exerted, duration of interaction, temperature, and participating substances, may be set with high precision, purity, and reproduceability.

All methods known per se for manipulating cells or particles, such as dielectrophoresis, sedimentation, centrifugation, hypoosmolar shock, exerting flow forces (e.g., mixing and shaking), filter techniques, optical manipulation using laser tweezers or mechanical manipulation, such as particle entrapment, and the like are usable as methods of bringing the cells into contact and coupling them to one another. The external forces are set so strongly that the cells remain in the state in which they are adhered to one another even when the force effect is deactivated or ended.

To modify antigen-presenting cells using diseased cells or cell components, such as e.g. membrane vesicles, which may advantageously be obtained easily from diseased cells, such as tumor cells, or cell components or particles, such as virus envelopes, both cell types are brought into an interaction region and subjected there to external forces to cause mutual contact. The forces to cause mutual contact are exercised as a function of the application until the desired chemical or physical bond has formed. If necessary, this is determined by observing or measuring the cells or the cell composite.

Single or multiple cells of both cell types may be caused to interact simultaneously.

After the contact, it is advantageous in some circumstances, as a function of the specific properties of the diseased cells, to reinforce the incorporation of the antigens in the cells to be modified through a field pulse, which leads to the reversible dielectric rupture of the membrane (e.g., field-induced endocytosis). A rupture pulse in hypoosmolar or isoosmolar solution is induced using an electrode device, for example, as is known per se for electroporation of cells. This has the advantage that membrane components may be absorbed especially rapidly from the cells to be modified.

According to a further embodiment of the present invention, the antigen-presenting cells are brought into contact with cell components of diseased cells, particularly with membrane components, in order to receive the antigens from them. The transfer of the antigens is performed in that membrane components of the diseased cells adhere to the antigen-presenting cells or are incorporated or absorbed into their membranes. For this purpose, the desired cell components are first separated from the nuclei and the cytoplasm of the diseased cells and then caused to interact with the antigen-presenting cells. Membrane fragments, which form spontaneously into membrane vesicles, are preferably used as cell components.

According to a further aspect of the present invention, a method of passive immunization or inoculation of organisms against illnesses, particularly tumor illnesses, is provided. To implement the method, allogenic, e.g., dendritic cells, i.e., cells of another (healthy) human, and, for example, tumor cells of the patient, against which the treated cells are to be immunologically activated, are removed. The cells removed are subjected to one of the methods cited for mutual interaction, so that the dendritic cells absorb tumor antigens. The treated dendritic cells are subsequently reinjected into the subject (patient). Already irradiated (killed) tumor cells may also be used as tumor cells. A passive immunization according to the present invention may also be performed analogously against bacterial or viral infections.

A subject of the present invention is comprising also a cellular vaccine that contains cells which are modified using antigens of diseased cells or cell components.

The object of the present invention comprises also devices for manipulating biological cells. A device according to the present invention contains a unit for storing and supplying cells or cell components into an interaction region (e.g., liquid container), a manipulation unit in the interaction region, e.g., a microelectrode system for dielectrophoretic manipulation of cells, possibly a measurement and observation unit for detecting the result of the cell treatment, and an extraction unit. For the modification of solid phase adsorbed cells described above, the interaction region contains a solid-phase substrate for cells which are to be modified or which are used for the modification of other cells.

The present invention is preferably used for passive immunization or inoculation against tumor growth.

Further details and advantages of the present invention are illustrated in the following description of embodiments with reference to the attached drawings.

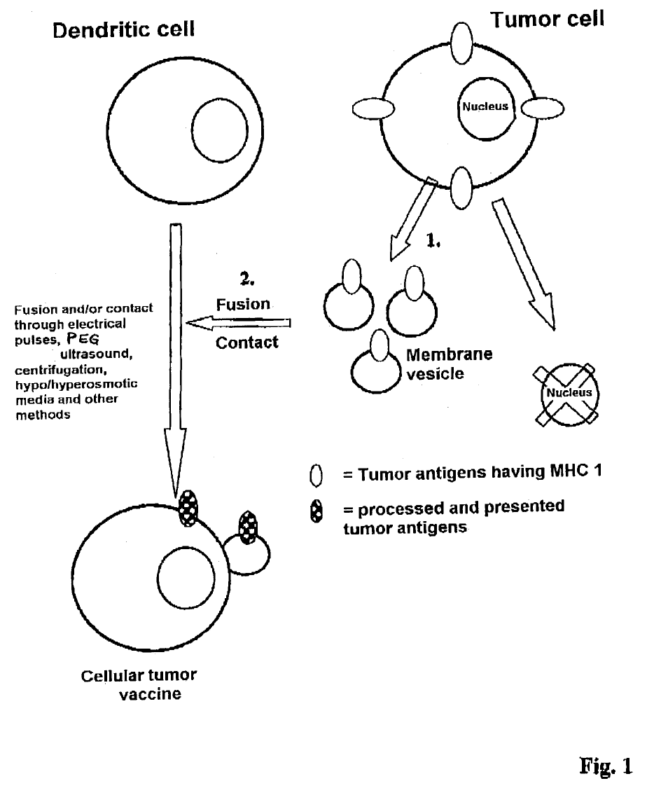

FIG. 1 shows a schematic illustration of the modification of dendritic cells according to the present invention, and

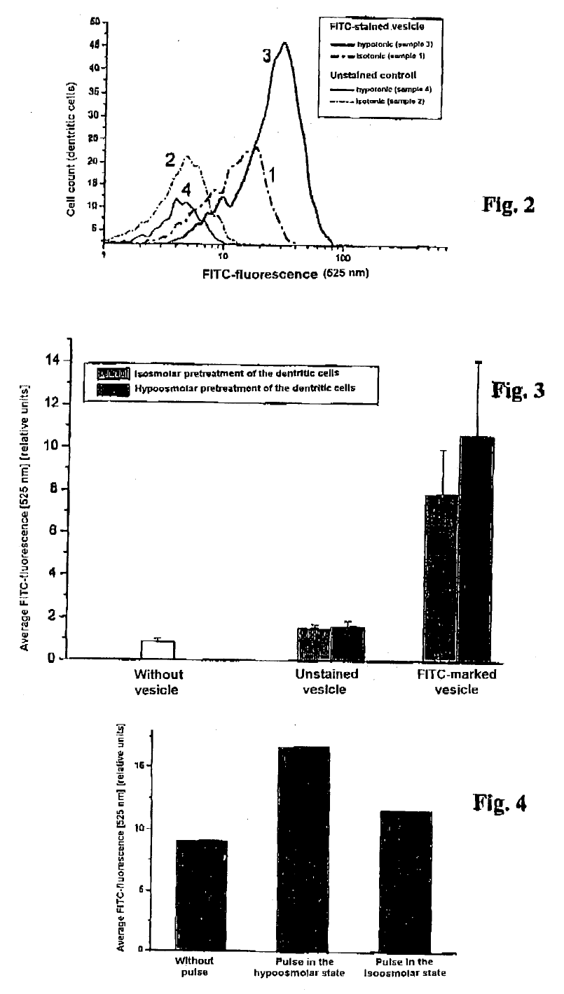

FIGS. 2 to 4 show graphic representations of experimental results which were achieved using cells modified according to the present invention,

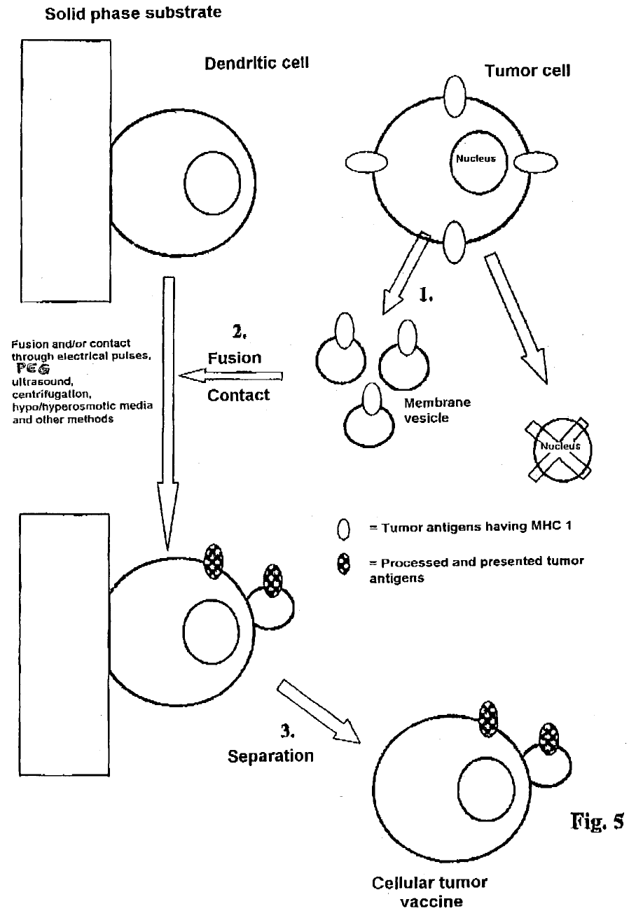

FIG. 5 shows a schematic illustration of the solid-phase modification of dendritic cells according to the present invention, and

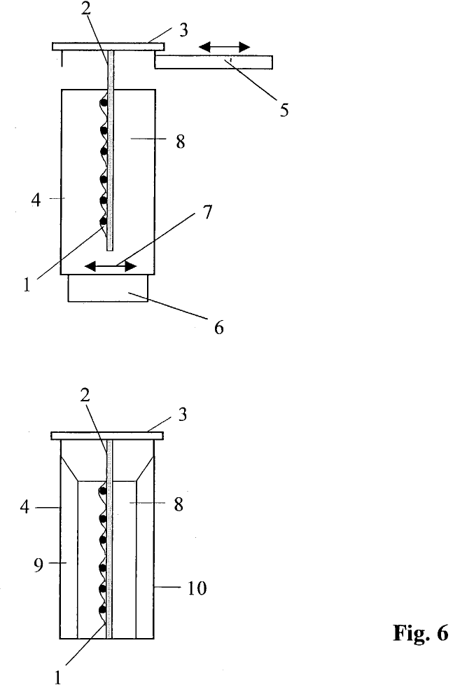

FIG. 6 shows schematic sectional views of devices according to the present invention.

The basic principle of the present invention, namely the incorporation of antigens into antigen-presenting cells through their contact with diseased cells or cell components under the effect of external forces, may be implemented in each case with the goal of immunotherapy using greatly varying types of diseased cells. According to the present invention, dendritic cells, T-cells, B-cells, or mast cells may particularly be used as antigen-presenting cells. The present invention is described in the following, without restriction, using the example of the modification of dendritic cells. All biological molecules to which an organism reacts with an immune answer are understood here as “antigens”. The antigens may be of natural or artificial origin. They may be transferred from cells, cell components, synthetic particles (such as membrane vesicles) or freely from the suspension onto the cells to be modified. In addition to tumor cells, bacteria, viruses/virus envelopes, stem cells, bone marrow cells, or epithelial cells, for example, may also be used in order to transfer antigens or membrane components of the cells onto the dendritic cells. In the following, the present invention is described without restriction in regard to the modification of dendritic cells, adsorbed onto a solid phase, using tumor cells.

Furthermore, the present invention may be implemented by coupling entire diseased cells, which, if necessary, were previously killed, or their membrane components onto the dendritic cells. The membrane components were either first obtained from the diseased cells and then caused to interact with the dendritic cells or transferred from the diseased cells onto the dendritic cells during the contact therewith. The interaction of the dendritic cells with the diseased cells has the advantage of a simplified method cycle, since the step of separate provision of the membrane components is dispensed with. However, special measures must be taken to protect against metastasis or infection, in case the diseased cells are completely coupled to the dendritic cells. If the membrane components are prepared separately, this has the advantage that the modified dendritic cells are hardly changed in their size and their functional properties. The modified cells move on the path to the lymph nodes and behave there like unmodified cells. Therefore, the immunostimulating function of the dendritic cells is improved in comparison to cell-cell fusion. In the following, without restriction, reference is predominantly made to the interaction of dendritic cells with membrane components which were prepared separately.

An essential feature of the present invention is that on the one hand the dendritic cells, which are freely suspended or adsorbed onto a solid phase, and on the other hand the diseased cells or their cell components are subjected to external forces in a shared suspension. This allows short-term contact, which, depending on the type of the external forces, may be in the range of microseconds or milliseconds, a few minutes, or up to one or two hours. A significant reduction of the treatment duration is thus achieved in relation to conventional methods. The proportion of the dendritic cells which survive the treatment without functional loss is thus elevated. The effectiveness of the vaccine according to the present invention on the basis of modified dendritic cells is elevated. In the following, without restriction, reference is made to the exertion of flow forces in the shared suspension, for example, through movement of the vessel containing the suspension or other types of mixing and/or electrical forces. The external forces may analogously be exerted through dielectrophoretic forces, centrifugation forces, optical forces, and/or the further forces cited above. In this case, the corresponding techniques, known per se, for handling cells or cell components are usable. Advantageously, large cell counts (10⁶ to 10⁸ or more) may be treated.

FIG. 1 schematically shows preparation step 1 for producing membrane vesicles from tumor cells and contacting step 2 for modifying the dendritic cells to illustrate the method according to the present invention for cell modification and suspensions. In the following, first the preparation step is described with reference to an exemplary method and then the contacting step is described with reference to various exemplary methods.

1 Preparation Step (Suspension or Solid-Phase Modification)

During the preparation step, membrane vesticles are produced from tumor cells using a homogenization method known per se with subsequent removal of the cell nuclei. The method is described, for example, by J. M. Graham et al. in “Molekularbiologische Mebrananalyse” [Molecular Biology Membrane Analysis], Spektrum Akademischer Verlag GmbH, 1998.

The tumor cells are homogenized using a glass Potter homogenizer. For example, 5 ml probes are homogenized multiple times (e.g., 15 times) using a typical laboratory Potter homogenizer (gap width, for example, 150 μm). Subsequently, the homogenized suspension is centrifuged. As a result of the centrifugation, the heavy cell components, particularly the nuclei and organelles, are located in the pellet. The cell components of lower density (membrane components) are located in the supernatant. Centrifugation is performed, for example, at 3500 RPM (2000 g) for a duration of 15 minutes. After the centrifugation, the supernatant is separated from the pellet. Membrane vesicles, i.e., closed membrane envelopes in spherical form, filled only with the suspension liquid, form from the membrane components. The membrane vesicles have a characteristic diameter of less than 1 μm. The tumor antigens having the immunostimulating MHCI complex are contained in the membranes of the membrane vesicles. The vesicles suspensions are stored, for example, as 4 ml samples in the refrigerator. The original osmolality of the vesicles suspensions is adjusted from an initial 20 mOsm to a higher osmolality (e.g., 80 mOsm) to avoid damage to the membrane vesicles through osmotic pressure. This is performed, for example, using tenfold PBS (80 μl tenfold PBS+4 ml vesicle suspension).

As a result of preparation step 1, the cell nuclei are separated from the membrane vesicle, as is schematically illustrated in FIG. 1. The modification of the dendritic cells according to the present invention is preferably performed using only the membrane vesicles which carry the tumor antigens.

For analysis or test purposes (see below for experimental results), staining of the membrane proteins may be provided. For staining, the tumor cell suspension first has the marking pigment (e.g., FITC) added to it. Subsequently, the unbound pigment is removed from the suspension. The cell suspension (1.2 ml, PBS, 1*10⁷/ml) has 50-150 μM FITC (36 μl, original solution 5 mM in DMF) added to it. The staining lasts approximately 5-15 minutes at 37° C. Multiple washing steps using a protein which binds the remaining pigment are performed to remove the unbound pigment (e.g., using PBS-BSA, 20° C., 1% BSA), each combined with centrifugation steps. Subsequently, washing in a buffer solution, a centrifugation, and the provision of the pellet having the tumor cells in PMSF buffer, which is hypoosmolar, so that the tumor cells swell, are performed. The following composition is provided as the PMSF buffer: 10 mM tris, 0.5 mM protease inhibitor PMSF (phenylmethylsulfonyl fluoride), pH 7.2.

2 Contacting Step (Suspension Modification)

Osmotically-Induced Incorporation of the Vesicles into Dendritic Cells

To cause contact, first a batch of the dendritic cells is provided as a cell suspension or as a cell sample (without the suspension liquid). The batch is then transferred with the vesicle suspension into a common suspension, in which the membrane vesicles are brought into contact with the dendritic cells under the effect of external forces. The suspension of dendritic cells may be made isotonic or hypotonic. The hypotonic suspension is preferred, since the dendritic cells are swollen therein. The experimental results described below show that the swollen dendritic cells are modifiable with the membrane vesicles with greater effectiveness. To produce the isotonic suspension, for example, a suspension of 10⁶ dendritic cells/ml (2 ml) is first centrifuged and then washed in 5 ml PBS (280 mOsm) and then received in 100 μl PBS (280 mOsm). To produce the hypotonic suspension, 2 ml of the starting suspension is correspondingly centrifuged and then washed in 5 ml PBS, diluted with H₂O (80 mOsm) and then received in 100 μl PBS (80 mOsm).

The isotonic or hypotonic suspension is then combined with the vesicle suspension (2 ml, 80 mOsm), in order to cause the modification of the dendritic cells with the membrane vesicles according to the present invention. For this purpose, first there is an incubation at 37° C. (10 minutes). Subsequently, an adjustment of the suspension to an isoosmolar state occurs (e.g., to 280 mOsm using 147 μl tenfold PBS). In this way, the dendritic cells are made smaller, and an irregularly curved membrane surface results, whose shape promotes the incorporation of the membrane vesicles into the membrane, endocytosis processes, and external adhesion. An incubation at 37° C. for approximately 1.5-2 hours follows. During this incubation, the modification of the dendritic cells occurs. The external forces are exerted in the form of flow forces. Subsequently, washing with PBS (280 mOsm) is performed, in order to remove the non-coupled (still free) membrane vesicles.

The suspension existing at the end contains modified dendritic cells as a cellular tumor vaccine (see FIG. 1, left bottom), which contains the antigens of the tumor cells directly in the membrane or in adhered membrane components.

FIG. 2 illustrates the fluorescence analysis of stained cell samples in comparison to unstained control samples using FACS analysis. For samples 1 and 3, an isotonic (sample 1) or hypotonic (sample 3) batch of dendritic cells were added to suspensions having stained vesicles. For control samples 2 and 4, there was a corresponding addition of unstained vesicles. It was shown that a strongly elevated intensity of the FITC fluorescence resulted at 525 nm in the dendritic cells after the incubation with the FITC-marked membrane vesicles of tumor cells (H7 cells). The pretreatment of the dendritic cells using hypotonic medium (sample 3) causes a significantly higher incorporation of membrane vesicles than the isotonic pretreatment (sample 1). The curves of control samples 2 and 4 show the significantly weaker autofluorescence of the dendritic cells after fusion with unstained vesicles.

A quantitative evaluation of the fluorescence analysis is illustrated in FIG. 3. The FITC fluorescence is used as a scale for the incorporation of the stained vesicles into the dendritic cells. The dendritic cells treated with unstained vesicles show a slight elevation of the autofluorescence. The stained vesicles provide significantly higher fluorescence intensities, the hypoosmolar pretreatment of the dendritic cells resulting in a stronger fluorescence, which confirms the more effective incorporation of the stained vesicles into the dendritic cells.

Electrically Induced Incorporation of the Vesicles into Dendritic Cells

For the electrically induced incorporation of the vesicles, first an isotonic or hypotonic suspension of dendritic cells was combined as described above with a vesicle suspension and incubated for short time (e.g., 5 minutes) at room temperature. 800 μl of the suspensions of both dendritic cells and vesicles were subjected to an electric field pulse, which produces the external forces to modify the dendritic cells. The parameters of the field pulse are, for example, 1 kV/cm, duration: 20 μs. Ten minutes after pulse application, the osmolarity of the hypotonic suspension was adjusted to 280 mOsm by adding 10 × PBS. After the application of the field pulse, an incubation was performed at room temperature and subsequently, to regenerate the cells, an incubation was performed at 37° C. for one hour. Finally, the samples were washed with PBS (280 mOsm), in order to remove the non-fused, free vesicles.

The FACS analysis of the modified dendritic cells is illustrated in FIG. 4. In FIG. 4, the average FITC fluorescence intensities are illustrated at 525 nm for the different samples. The left column shows the fluorescence without application of the field pulse. This sample therefore corresponds to the method of osmotically-induced vesicle incorporation described above. The middle column shows that the pulse application causes almost doubling of the vesicle incorporation in the hypoosmolar state of the cell suspension. For pulse application in the isoosmolar state (right column) a less strong increase of the fluorescence results. This is explained in that the permeability of the membrane surface is lower in the isoosmolar state than in the hypoosmolar state.

The cell modification according to the present invention using solid-phase adsorbed cells is schematically illustrated in FIGS. 5 and 6. In addition to preparation step 1 and contacting step 2, a separation step 3 for detaching the modified dendritic cells from the solid-phase substrate, for example, is provided. Preparation step 1 is performed according to the procedure described above. Contacting and separation steps 2 and 3 are implemented as follows.

Contacting Step 2/Separation Step 3 (Solid-Phase Modification)

For contacting, first a solid-phase substrate having adsorbed dendritic cells is provided. The solid-phase substrate is then transferred with the vesicle suspension into a shared suspension, in which the contact of the membrane vesicles with the dendritic cells under the action of external forces occurs as shown in FIG. 5.

The dendritic cells removed from the body of the organism to be treated are arranged as a monolayer or submonolayer on the solid-phase substrate. This is performed by a suitable immobilization method. The immobilization includes, for example, mechanical application of the cells onto the solid phase, possibly using binding layers on the solid-phase surface. Cells may be suctioned onto a filter membrane through a partial vacuum (analogously to filtration). For the binding layers, fibronectin, collagen, polylysine, gelatins, matrigel, FCS (fetal calf serum), or alginate are used, for example. Plastic or glass substrates are used, for example, as a solid-phase substrate. Preferably, the dendritic cells are immobilized on microporous plastic membranes. PET or PC membranes (thicknesses approximately 15 to 25 μm, pore sizes approximately 0.4 to 1 μm, porosity approximately 15 to 20%) are, for example, used as membranes. For the substrates, each cell preferably covers a pore, alternatively even multiple pores, in the adhered state.

The solid-phase substrate is positioned with the cells in a device according to the present invention, whose details are described in the following with reference to FIG. 6, in an interaction region together with the suspension of diseased cells or cell components, for example, the membrane vesicles described above. The transfer of the antigens to the dendritic cells occurs in the interaction region. The transfer is performed through osmotically-induced or electrically-induced incorporation of the vesicles into the dendritic cells, analogously to the procedures described above in connection with the suspension modification.

To exert external forces in the form of flow forces, the membrane with the dendritic cells and/or the suspension liquid in the interaction region is/are moved. The movement is preferably performed in such a way that turbulent flows arise in the interaction region near the surface of the solid-phase substrate, due to which the particles located in the suspension flow as numerously as possible and with the highest possible forces against the solid-phase substrate.

For the electrically-induced incorporation, for example, 800 microliters of the suspension having the vesicles and the immersed solid-phase substrate are subjected to an electric field pulse, which produces the external forces for modifying the dendritic cells. The application of the field pulse is also preferably performed in this case using an electrode unit of the device according to the present invention (see FIG. 6). Alternatively to applying the field pulse, the vesicles may also be guided dielectrophoretically to the solid-phase adsorbed dendritic cells. The external forces cited are produced by the polarization forces produced under the effect of high frequency electric fields. Subsequently, washing with PBS (280 mOsm) is performed, in order to remove the non-coupled (still free) membrane vesicles.

The solid-phase substrate now provided carries modified dendritic cells, which are detached from the solid-phase substrate during separation step 3 to provide the cellular tumor vaccine (see FIG. 5, right bottom). The tumor vaccine contains the antigens of the tumor cells directly in the membrane or in adhered membrane components.

The separation of the modified dendritic cells from the solid-phase substrate may be performed through suitable detachment techniques, known per se. For example, it is possible to perform the separation through incubation in a hypoosmolar buffer solution or through enzymatic degradation.

Embodiments of a Device for Solid-Phase Modification of Antigen-Presenting Cells

FIG. 6 shows two embodiments of devices according to the present invention, which are differentiated by the type of force exerted during the interaction between adsorbed dendritic cells and the suspended particles. In both cases, dendritic cells 1 are positioned on solid-phase substrate 2, which is attached to carrier 3 using a frame (not shown). Carrier 3 is positioned using a support in such a way that solid-phase substrate 2 projects into a liquid or suspension container (e.g., cuvette 4).

To exert flow forces, a pivot device 5, using which carrier 3 may be moved, and/or a stirring device 6, 7 are provided, using which suspension liquid 8 may be moved inside cuvette 4. Stirring device 6, 7 is formed by a magnetic stirrer, for example.

For electrically-induced incorporation of the vesicles into the adsorbed dendritic cells, as shown in the lower part of FIG. 6, an electrode unit 9, 10 is provided in the inside of cuvette 4. The electrode unit comprises, for example, metallic coatings (e.g., made of platinum), which are positioned on the inside of cuvette 4 and are electrically connected to a control device (not shown). Solid-phase substrate 2 having cells 1 projects into the intermediate space between electrodes 9, 10, which is filled with suspension liquid 8. Carrier 3 lies on the upper edge of cuvette 4.

The device according to the present invention is also equipped with liquid supply units, temperature control units, and manipulators, which are provided depending on the application and are not illustrated in FIG. 6. The device according to the present invention may also be produced as a flow-through system, in which modification of cells according to the present invention occurs continuously with continuous supply of diseased cells or cell components or antigen-presenting cells.

For cell modification in suspensions, the device is correspondingly constructed without the solid-phase substrate.

Important Features of the Present Invention are Summarized in the Following:

a) The inventors have determined that passive immunization or inoculation may surprisingly be achieved without fusion of dendritic cells with diseased cells. It is sufficient if the cells are brought into contact. Close contact between cells of both cell types may be achieved with particular advantage, through chemical bonds or through physical forces such as dielectrophoresis, centrifugation, filter techniques, etc., if the dendritic cells are in an adsorbed state on a solid-phase substrate. The contacting is preferably to be performed in such a way that the contacted cells do not detach from one another when the mechanical and/or electrical forces are deactivated.

b) It is sufficient if the membrane of the tumor cells is brought into close physical or chemical contact with the dendritic cells and/or if irradiated (killed) tumor cells are used.

c) It is particularly advantageous to perform the contacting via dielectrophoresis in microstructures. This particularly has the advantage that high fields are achieved locally in microelectrode systems.

d) It is advantageous to provide an absorption of adhering tumor membrane pieces in the dendritic cells and to elevate this by using strongly hypoosmolar solutions. This results from endocytosis possibly having been observed after transfer in isoosmolar solutions. The use of isoosmolar solutions is, however, also possible in principle.

e) It is particularly advantageous to trigger a field-induced endocytosis. This means that after physical or chemical contact, a rupture pulse is induced in the hypoosmolar solution.

1. A method of modifying antigen-presenting cells, in which the antigens of diseased cells are transferred to the antigen-presenting cells, characterized in that
the antigen-presenting cells are brought into contact with the diseased cells or their cell components by introduction into a shared suspension and the exertion of external forces, wherein membrane components having antigens without cell nuclei from the diseased cells or the cell components being transferred onto the antigen-presenting cells and a separation of the modified antigen-presenting cells from free diseased cells or cell components in the suspension being performed.

2. The method according to claim 1, wherein the antigen-presenting cells and the diseased cells are incorporated into the shared suspension while freely suspended.

3. The method according to claim 1, wherein the antigen-presenting cells are positioned on a solid-phase substrate and the diseased cells or their cell components are incorporated into the shared suspension while freely suspended, detachment of the modified cells from the solid-phase substrate being performed after the transfer of the membrane components.

4. The method according to one of the preceding claims, wherein the antigen-presenting cells are brought into contact with the diseased cells or the cell components by exerting flow forces, for example, stirring or shaking, dielectrophoretic forces, centrifugation forces, filter techniques, sedimentation, hypoosmolar shock, and/or optical forces and chemical bonds.

5. The method according to claim 2, wherein the antigen-presenting cells are brought into contact with the diseased cells or the cell components through pipetting into the suspension and production of the flow forces through mixing.

6. The method according to one of the preceding claims, wherein the suspension is produced in an isotonic solution.

7. The method according to one of claims 1 to 5, wherein the suspension is produced in a hypotonic solution.

8. The method according to one of the preceding claims, wherein the antigen-presenting cells and the diseased cells or the cell components are subjected to a field treatment.

9. The method according to claim 8, wherein the field treatment includes the application of at least one electrical high-voltage pulse and/or the application of polarization forces under the effect of dielectrophoresis.

10. The method according to one of the preceding claims, wherein the cell components include membrane vesicles which are produced from membrane parts of the diseased cells.

11. The method according to claim 10, wherein the membrane vesicles are produced through homogenization and centrifugation of the diseased cells.

12. The method according to one of the preceding claims, wherein the antigen-presenting cells include dendritic cells, T-cells, B-cells, or mast cells.

13. The method according to one of the preceding claims, wherein the diseased cells or cell components include tumor cells, epithelial cells, stem cells, bone marrow cells, or virus envelopes.

14. The method according to one of the preceding claims, wherein there is short-term contact of the dendritic cells with the diseased cells or their cell components.

15. The method according to claim 1, wherein at least one cell species is moved using external forces toward the other species until they come into mutual contact.

16. The method according to claim 1, wherein the external forces are exerted in such a way that the cells are pressed against one another.

17. The method according to claim 1, wherein an exchange of substances or cell components occurs between the antigen-presenting cells, particularly dendritic cells, and the diseased cells or their cell components.

18. The method according to one of the preceding claims, wherein an endocytosis, induced by an electric field, is performed.

19. A cellular tumor vaccine, which contains antigen-presenting cells, which were modified according to a method according to one of the preceding claims.

20. A cellular tumor vaccine, which contains antigen-presenting cells, into whose cell membranes antigens or antigen-carrying membrane components of diseased cells, particularly tumor cells, are incorporated.

21. A device for modifying antigen-presenting cells, particularly dendritic cells, using diseased cells or their cell components, which includes:
a device for providing a suspension, in which the antigen-presenting cells and the diseased cells or their cell components are positioned,
a device for exerting external forces to bring the suspension components into contact, and
an extraction device for obtaining the modified antigen-presenting cells.

22. The device according to claim 21, wherein the device for providing the suspension includes a suspension container.

23. The device according to claim 1, wherein a solid-phase substrate, which is positioned using a carrier (3) in a liquid container (4) for receiving a suspension of diseased cells or their cell components, is provided for receiving adhered antigen-presenting cells.

24. The device according to one of claims 21 to 23, which has a homogenization and centrifugation device for producing nucleus-free membrane vesicles of the diseased cells.

25. The device according to one of claims 21 to 24, wherein the unit for exerting external forces includes a pivot device (5) for moving the carrier (3) relative to liquid container (4) and/or a stirring device (6, 7).

26. The device according to one of claims 21 to 24, wherein the unit for exerting external forces includes an electrode unit for exerting an electric field pulse in the inside of the liquid container (4).
